(19) United States
(12) Patent Application Publication
(10) Pub. No.: US 2015/0037897 A1
(43) Pub. Date: Feb. 5, 2015

(54) METHOD OF ANALYZING A SAPPHIRE ARTICLE

(71) Applicant: GTAT Corporation, Merrimack, NH (US)

(72) Inventors: Gopal Prabhu, San Jose, CA (US); Venkatesan Murali, San Jose, CA (US); Daniel Squiller, San Diego, CA (US)

(21) Appl. No.: 14/451,915

(22) Filed: Aug. 5, 2014

Related U.S. Application Data
(60) Provisional application No. 61/862,242, filed on Aug. 5, 2013.

(51) Int. Cl. C30B 33/10; G01N 33/24; C30B 29/20; C23F 1/30; C30B 11/00

(52) U.S. Cl. CPC C30B 33/10; C23F 1/30; C30B 11/003; C30B 29/20; G01N 33/24
USPC 436/73; 216/102; 216/103; 216/38; 117/81

(57) ABSTRACT

A method of removing material from a sapphire article is described. In particular, the method comprises the step of providing an initial sapphire layer and reducing the thickness of the layer while not significantly increasing the surface roughness of the layer, Cover plates for electronic device and methods of preparing them are also disclosed, along with a method of analyzing a sapphire article produced by the present method.

### CROSS-REFERENCE TO RELATED APPLICATION

The present application claims the benefit of U.S. Patent Application No. 61/862,242 filed Aug. 5, 2013, which is incorporated herein by reference.

### BACKGROUND OF THE INVENTION

1. Field of the Invention

The present invention relates, in general, to a method of removing material from a the surface of an initial sapphire article, and to uses and applications of the resulting sapphire article.

2. Description of the Related Art

There are many types of mobile electronic devices currently available which include a display window assembly that is at least partially transparent. These include, for example, handheld electronic devices such media players, mobile telephones (cell phones), personal data assistants (PDAs), pagers, tablets, and laptop computers and notebooks. The display screen assembly may include multiple component layers, such as, for example, a visual display layer such as a liquid crystal display (LCD), a touch sensitive layer for user input, and at least one outer cover layer used to protect the visual display. Each of these layers are typically laminated or bonded together.

Many of the mobile electronic devices used today are subjected to excessive mechanical and/or chemical damage, particularly from careless handling and/or dropping, from contact of the screen with items such as keys in a user's pocket or purse, or from frequent touch screen usage. For example, the touch screen surface and interfaces of smartphones and PDAs can become damaged by abrasions that scratch and pit the physical user interface, and these imperfections can act as stress concentration sites making the screen and/or underlying components more susceptible to fracture in the event of mechanical or other shock. Additionally, oil from the use's skin or other debris can coat the surface and may further facilitate the degradation of the device. Such abrasion and chemical action can cause a reduction in the visual clarity of the underlying electronic display components, thus potentially impeding the use and enjoyment of the device and limiting its lifetime.

Various methods and materials have been used in order to increase the durability of the display windows of mobile electronic devices. For example, polymeric coatings or layers can be applied to the touch screen surface in order to provide a barrier against degradation. However, such layers can interfere with the visual clarity of the underlying electronic display as well as interfere with the touch screen sensitivity. Furthermore, as the coating materials are often also soft, they can themselves become easily damaged, requiring periodic replacement or limiting the lifetime of the device.

Another common approach is to use more highly chemically and scratch resistant materials as the outer surface of the display window. For example, touch sensitive screens of some mobile devices may include a layer of chemically-strengthened alkali aluminosilicate glass, with potassium ions replacing sodium ions for enhanced hardness, such as the material referred to as Gorilla® glass available from Corning. However, even this type of glass can be scratched by many harder materials, including metal keys, sand, and pebbles, and, further, as a glass, is prone to brittle failure and shattering.

Sapphire has also been suggested and used as a material for either the outer layer of the display assembly or as a separate protective sheet to be applied over the display window. However, sapphire is relatively expensive, particularly at the currently available thicknesses, and reducing a layer of sapphire to a more desirable thickness adds considerable cost and time. For example, typically, a sapphire layer, such as a wafer, is removed from a larger sapphire material, such as a boule, using a wire saw, and the resulting sapphire must then undergo a series of coarse and fine grinding steps in order to reduce the thickness to the desired value. Such grinding steps are expensive, time consuming, and introduce significant surface damage that must be removed using a series of polishing steps in order to produce a transparent layer, further adding cost and time to the process.

Thus, while sapphire materials are available which can enable the display of a mobile electronic device to be relatively resistant to damage, there remains a need in the industry for low cost methods for producing thin layers of transparent sapphire.

### SUMMARY OF THE INVENTION

The present invention relates to a method of producing a sapphire article. The method. comprises the steps of providing an initial sapphire article having a thickness and at least one surface, the surface of the initial sapphire article having an average surface roughness value of Ra_{I}; and reducing the thickness or size of the initial sapphire article by contacting the surface with a reagent solution to produce the sapphire article which has a thickness that is less than the thickness of the initial sapphire article and further has a final surface having an average surface roughness value of Ra_{F}, wherein (Ra_{F}−Ra_{I})/Ra_{I} is less than or equal to 0.2. Thus the method reduces at least one dimension of a sapphire article without significantly increasing the roughness of the surface of the article.

The present invention further relates to a method of analyzing a sapphire article comprising the steps of producing a sapphire article by the method of the present invention and subsequently analyzing the resulting sapphire article. Preferably the analysis step comprises determining the crystal quality of the sapphire article.

It is to be understood that both the foregoing general description and the following detailed description are exemplary and explanatory only and are intended to provide further explanation of the present invention, as claimed.

### BRIEF DESCRIPTION OF THE DRAWINGS

FIG. 1 shows a prior art method of reducing the thickness of a sapphire layer.

FIG. 2 shows a specific embodiment of the method the present invention.

### DETAILED DESCRIPTION OF THE INVENTION

The present invention relates to a method of removing sapphire from at least one surface of an initial sapphire article, such as a sapphire layer, thereby reducing at least one dimension, such as the thickness, and forming a final sapphire article.

The method of the present invention is a method of preparing a sapphire layer having a desired or targeted thickness. The method comprises the steps of providing an initial sapphire layer having a starting thickness and then contacting at least one surface of that layer with a reagent solution to reduce the overall thickness, thereby producing the desired final sapphire layer having the targeted thickness. Thus, the thickness of the desired final sapphire layer is less than the thickness of the initial sapphire layer and is chosen based on the targeted use of the thinned layer. Preferably, the final sapphire layer has a thickness of less than about 2 mm, such as less than about 1 mm and less than about 0.8 mm. As a particular example, the initial sapphire layer has a thickness of from about 0.35 mm to about 0.75 mm. Such layers of sapphire would be particularly useful as a protective layer for various electronic devices, described in more detail below.

In order to achieve these desired final thicknesses, the initial sapphire layer must therefore be thicker, and the thickness of the initial layer can vary depending, for example, on the method used to prepare the initial layer as well as based on the overall costs of the removal process. For example, the initial sapphire layer can have a thickness of less than about 5 mm, such as less than about 2 mm and less than about 1 mm. As a particular example, the initial sapphire layer has a thickness of from about 0.4 mm to about 0.8 mm, Preferably, in order to reduce cost and prevent waste, the initial sapphire layer is not so much thicker than the final desired sapphire layer that excessive removal of sapphire material is required. For example, preferably the initial sapphire layer is not more than 50% thicker than the final sapphire layer, including not more than 40% thicker and not more than 30% thicker. More preferably, the initial sapphire layer is less than 25% thicker than the final sapphire layer, such as between about 25% and about 5% thicker.

The initial layer of sapphire used in the method of the present invention can be any sapphire layer known in the art. For example, the sapphire layer can be a wafer, such as a circular, oval, square or rectangular wafer having one or more sides and a top and bottom surface, with a thickness as described above. Furthermore, the sapphire layer can have any crystal orientation. As is known in the art, sapphire may include one of several different crystalline axes, such as the c-axis, the m-axis, or the a-axis, and the properties of a sapphire layer can vary depending on this crystal orientation. The initial sapphire layer used in the present method can have any orientation relative to a surface of the layer. For example, the sapphire layer having an initial surface may have a c-axis orientation in a direction perpendicular to that surface, Alternatively the layer can have an a-axis orientation. Choice of crystal orientation can depend on how the sapphire material is prepared.

The initial sapphire layer can be prepared using a variety of known techniques. For example, the initial sapphire layer can be prepared by cutting or slicing a layer from a donor sapphire material, such as a boule or a portion of a boule. As a particular example, a boule of sapphire may be cored to remove a cylindrical section which can then be sliced or cut into wafers using a saw, such as a diamond wire saw. The cored section can have a defined crystal orientation, depending on how the donor material was prepared (a-axis, c-axis, or m-axis, for example). The resulting layer can be mechanically ground to a desired thickness and optionally further polished if needed to remove any unwanted surface defects. Such a method is particularly useful for relative thick sapphire layers, including those having a thickness of greater than about 0.100 mm, although thinner sapphire layers can also he produced by this method.

As another example, an initial sapphire layer having a thickness of less than about 100 microns can be prepared using various layer transfer methods known to remove thin layers from a donor sapphire material, including, for example, controlled spalling or ion implantation and exfoliation method, such as the ion implantation/exfoliation method generally described in U.S. patent application Ser. No. 12/026,530 entitled, “Method to Form a Photovoltaic Cell Comprising a Thin Lamina”, filed Feb. 5, 2008 and published as U.S. Patent Application Publication No. 2009/0194162 and U.S. patent application Ser. No. 13/331,909 entitled, “Method and Apparatus for Forming a Thin Lamina”, filed Dec. 20, 2011, both of which are incorporated in their entireties by reference herein, for the fabrication of a photovoltaic cell comprising a thin semiconductor lamina formed of non-deposited semiconductor material. Such an ion implantation/exfoliation method would be more advantageous over current methods of preparing thin wafers by sawing or cutting since the very properties considered desirable about sapphire (hardness and strength) can make it very difficult, time consuming, and costly to cut, grind, and optionally polish. in addition, sawing or cutting methods produce significant kerf losses, wasting valuable material, and cannot reliably be used to produce thin sapphire lamina.

The donor sapphire material used in either embodiment can be produced using any method known in the art. For example, the donor sapphire material can be prepared in a crystal growth apparatus, which is a high-temperature furnace capable of heating and melting a solid feedstock, such as alumina, in a crucible at temperatures generally greater than about 1000° C., such as greater than about 2000° C., and subsequently promoting resolidification of the resulting melted feedstock material to form a crystalline material, such as a sapphire boule. Preferably, the sapphire is prepared in a heat exchanger method crystal growth furnace, in which a crucible comprising alumina feedstock and at least one single crystal sapphire seed is heated above its melting point to melt the feedstock without substantial melting of the seed, and heat is then removed from the crucible using a heat exchanger, such as a helium-cooled or water-cooled heat exchanger, provided in thermal communication with the bottom of the crucible and positioned under the seed. This method has been shown to produce large, high quality sapphire boules from which the sapphire can be readily removed using available methods.

In the method of the present invention, the initial sapphire layer is reduced in thickness to produce a final sapphire layer by contacting at least one surface of layer with a reagent solution that removes sapphire material from the surface, Any of the surfaces of the initial sapphire layer can be contacted by the reagent solution. Also, multiple surfaces can be treated simultaneously or consecutively. For example, for a circular sapphire wafer having an edge and a top and a bottom surface, either the top or bottom surface, or both, may be contacted. with the reagent solution. As another example, the sapphire layer can be a multilayer wafer comprising a top sapphire layer having an accessible top surface and a bottom surface in contact with an additional layer, such as a glass layer, a polymer layer, or another sapphire layer. For this example, the accessible top surface of the initial sapphire layer can be contacted with the reagent solution.

The reagent solution can comprise any component capable of removing material from a sapphire layer. Preferably the reagent solution is an aqueous solution comprising one or more acids, such as sulfuric acid or phosphoric acid, although additional solvents or cosolvents may be present. More preferably, the reagent solution comprises a combination of sulfuric acid and phosphoric acid. Such combinations are known for etching the surface of sapphire, but have not been shown to be capable or even desirable for use in reducing the overall thickness of an entire layer or article of sapphire. The concentration of the reagent solution as well ratio of components (such as acids) can be adjusted depending on the type of sapphire layer, the desired rate of removal, and the conditions used for reducing the thickness of the layer.

Contact of the initial sapphire layer surface with the reagent solution can be made using any method known in the art. For example, reagent solution can be placed over the entire surface under specified conditions, described in more detail below, thereby causing removal of material from the surface and reducing the thickness of the layer. The more even the contact with the reagent solution, the more evenly the thickness is reduced. As another example, the sapphire wafer may be dipped or otherwise submerged in a bath including the reagent solution.

Once the surface of the initial sapphire layer is contacted by the reagent solution, removal of material can be assisted by mechanical or physical means, such as by rubbing, polishing, or grinding. However, for the present invention, it has surprisingly been found that such assistance is not required to reduce the thickness of the initial sapphire layer. Therefore, in a preferred embodiment of the method of the present invention, the surface of the initial sapphire layer is contacted with the reagent solution and the thickness of the layer is reduced without mechanical assistance. For this preferred embodiment, only the reagent solution is used to remove or dissolve the sapphire layer surface under controlled conditions, thereby reducing the overall thickness of the layer. Thus, for example, for this preferred embodiment, the thickness of the initial sapphire layer is reduced by contacting the layer, specifically a surface of the layer, with a solution comprising at least one active reagent and is preferably not a dispersion of any reagents. As such, the reagent solution is not any of the chemical mechanical polishing compositions known in the art and does not contain any solid material dispersed therein specifically provided to assist in the removal of material from the sapphire surface, such as an abrasive.

The conditions under which the surface of the initial sapphire layer is contacted and the thickness is reduced can be varied depending, for example, on the desired rate of reduction and on the type of equipment used (which depends, in part, on the size and shape of the sapphire layer). Preferably, the surface is contacted at a temperature of greater than or equal to about 200° C., such as greater than or equal to about 250° C. and greater than or equal to about 300° C. For example, the thickness of the initial sapphire layer can be reduced at a temperature from about 250° C. to about 350° C. by contacting with a reagent solution as described above. The pressure can be adjusted depending on the solvent used, For example, the reagent solution can be an aqueous solution, and thus, to attain the preferred temperatures, the surface would be contacted at a pressure that is higher than atmospheric pressure. These temperature conditions have been found to be able to reduce the thickness of the sapphire layer at a surprisingly fast rate. For example, it has been found that, at these temperatures, the thickness of the initial sapphire layer can he reduced at a rate of greater than or equal to about 10 microns/hour, including greater than or equal to about 20 microns/hour, 30 microns/hour, or 40 microns/hour. Other conditions may also he used to produce these rates of reduction, such as by adjusting the concentration and/or type of components of the reagent solution, given the benefit of the present disclosure.

While the thickness of the initial sapphire layer is reduced by the method of the present invention, surprisingly it has been found that this method does not significantly increase the surface roughness of the layer. In particular, if the surface of the initial sapphire layer has an average surface roughness value of Ra_{I} and the final surface of the sapphire layer after contacting with the reagent solution has an average surface roughness value of Ra_{F}, the difference in roughness between these surfaces is less than or equal to about 20%, Thus, (Ra_{F}−Ra_{I})/Ra_{I} is less than or equal to 0.2. Preferably, (Ra_{F}−Ra_{I})/Ra_{I} is less than or equal to 0.1 and more preferably is less than or equal to 0.05. Most preferably, (Ra_{F}−Ra_{I})/Ra_{I} is less than or equal to 0. Thus, the average surface roughness value most preferably does not increase but rather remains unchanged or decreases as a result of reducing the layer thickness by the method of the present method.

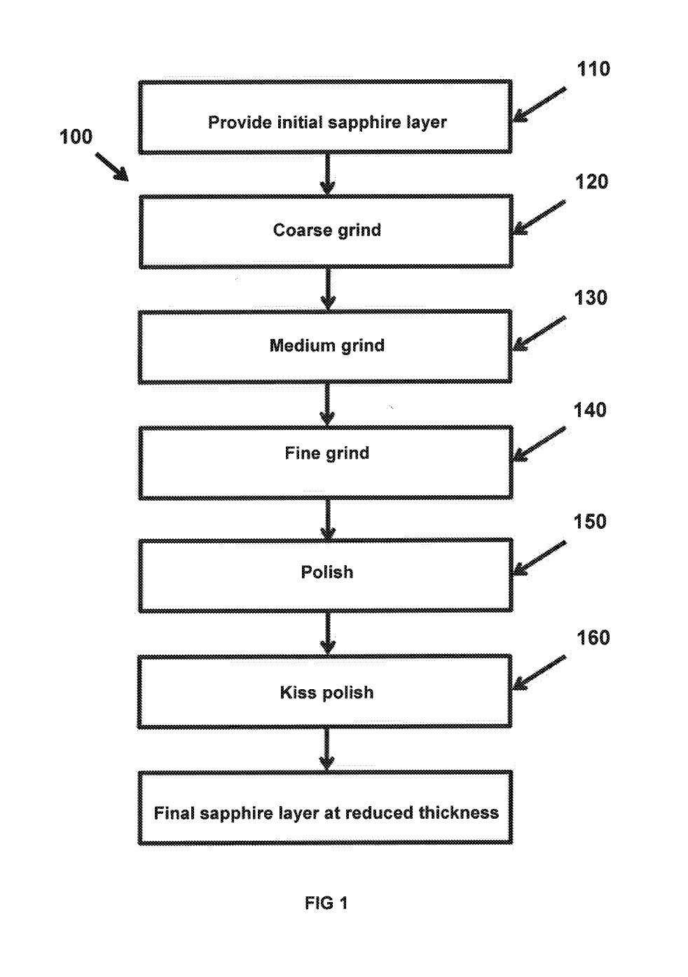

This is particularly surprising based on currently known methods of reducing the thickness of a sapphire layer. For example, as shown in FIG. 1, one current method 100 comprises first step 110 in Which an initial sapphire layer is provided followed by a series of grinding steps, such as coarse grind step 120, medium grind step 130, and fine grind step 140. Additional grinding steps can also be included, depending on such factors as the starting thickness of the initial sapphire layer and the conditions used for grinding. These grinding steps are generally slow. For example, fine grind step 140 is generally 1-3 microns/hour, which is needed to avoid large scale surface damage. Furthermore, the average surface roughness values resulting from these steps are also generally high, such as 2-5 A, which has a significant impact on the transparency and optical quality of the sapphire layer. Thus, in order to remove the significant amount of surface damage resulting from these grinding steps and provide a layer having the desired transparency, current method 100 also includes one or more polishing steps, 150, followed by final polish step 160 (sometimes referred to as a kiss polish). These grinding and polishing steps are extremely time consuming and dramatically increase the cost of the final sapphire having the desired overall thickness.

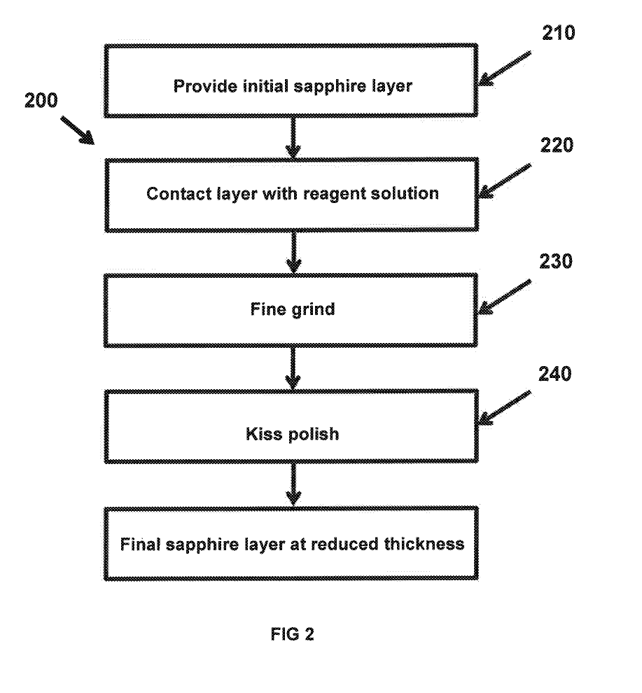

By comparison, as shown in FIG. 2, method 200, which is a specific embodiment of the method of the present invention, comprises first step 210 in which an initial sapphire layer is provided followed by contact step 220 in which one surface of the initial sapphire layer is contacted by a reagent solution. The thickness of the initially sapphire layer is reduced in step 220 without significantly increasing the surface roughness of the contacted surface. As shown in FIG. 2, this embodiment of the method can further include optional fine grinding step 230 and/or final polish step 240, which are similar to steps 140 and 160 of current method 100, in order to provide additional smoothness to the sapphire surface. As can be clearly seen, method 200 provides a significant improvement over current method 100. The overall process is far less complex, requiring fewer steps, less equipment, and significantly less time, and dramatically reduces the overall cost for producing a sapphire layer having a desired thickness and transparency.

The sapphire layer produced by the method of the present invention can be used in a variety of different applications. In particular, the sapphire layer can be used as a cover plate for an electronic device. Thus, the present invention further relates to a method of producing a cover plate configured for use with an electronic device, as well as to the cover plate produced, The cover plate has at least one transparent display region through which an image can be displayed, such as from a display element upon which the cover plate is placed. Non-transparent regions may also be present, particularly as decorative elements such as borders or as elements to delineate various functional sections of the display. The cover plate further comprises one or more sapphire layers, and the method of producing the cover plate comprises producing at least one of the sapphire layers by the method described in more detail above, followed by forming the cover plate comprising the resulting sapphire layer.

The cover plate formed in the method of the present invention can comprise one or more sapphire layers or laminae. The thickness of the sapphire layer can vary depending on the desired properties of the cover plate as well as the number of layers present, including any of the thicknesses described above relating to the final sapphire layer. More particularly, for the cover plate formed in the method of the present invention, the sapphire layer can have a thickness of from about 50 microns to about 2000 microns, including, for example, from about 50 microns to about 1000 microns, from about 50 microns to about 750 microns, from about 50 microns to about 600 microns, from about 100 microns to about 600 microns, from about 200 microns to about 600 microns, and from about 400 microns to about 600 microns. Thus, the cover plate may be a single, free-standing sapphire layer or may comprise multiple layers, at least one of which has a thickness in these ranges or may also comprise more than one sapphire layer or lamina having a thickness in these ranges, including 2-10 layers, such as 2-5 layers. For example, the cover plate may be a single, free-standing sapphire multilayer composite, wherein in each layer has a thickness of from about 400 microns to about 600 microns. Preferably, the sapphire layer is the exterior layer of the cover plate and the electronic device. The overall thickness of the cover plate of the electronic device of the present invention can vary depending on a variety of factors, including, for example, the number of layers, the desired size of the transparent display region, and the size of the device. In general, the cover plate has a thickness that is less than about 5 mm, such as less than about 3 mm, for a multilayer cover plate.

The cover plate may comprise a sapphire layer combined with one or more permanent or temporary carrier substrates or layers that provide additional desirable features to the cover plate. For example, the cover plate may further comprise a transparent layer affixed to the sapphire layer. The transparent layer can be any transparent material known in the art including, for example, a layer comprising glass, such as soda-lime, borosilicate, or aluminosilicate glass, including chemically-strengthened alkali aluminosilicate glass (such as the material referred to as Gorilla® glass available from Corning), or a layer comprising a polymeric material, such as a polycarbonate or a polymethacrylate such as polymethyl methacrylate (PMMA). The sapphire layer and the transparent layer may be combined using any technique known in the art, forming an interface in between, including the methods described in U.S. patent application Ser. No. 12/980,424 entitled, “A Method to Form a Device by Constructing a Support Element on a Thin Semiconductor Lamina”, filed Dec. 10, 2010, now U.S. Pat. No. 8,173,452, incorporated in its entirety by reference herein. For example, the interface may be formed by bonding with an adhesive layer, thereby affixing the sapphire layer to the surface of the transparent layer. Examples of suitable adhesives include, but are not limited to, polymers or combinations of polymers such as poly(propylene carbonate) (PC), poly(ethylene carbonate) (PEC), or poly(butylenes carbonate) (PBC). Electrostatic adhesion may also be used. In addition, the interface may be formed by thermally bonding the sapphire lamina to the transparent layer, such as through thermal compression bonding at, for example, pressures of from about 5-100 psi, including 40 psi, and temperatures from about 300-500° C., including 400° C. Specific bonding conditions would vary depending on the specific type of transparent layer used. Furthermore, the transparent layer may be fused or melted to the sapphire layer to form an interface, and the temperature will depend on the type of material used as the transparent layer. For example, temperatures for melting a glass substrate to the sapphire may be on the order of 650-1050° C. while lower temperatures, such as 110-150° C., would be suitable if the substrate is plastic.

In one embodiment, the transparent layer is a subsurface layer having a front or exterior-facing surface to which the sapphire layer is attached, thereby forming a multilayer composite. The subsurface layer can be thicker or thinner than the sapphire layer, depending on its purpose. For example, the subsurface layer can be relatively much thicker than the sapphire layer in order to provide improved strength, particularly when the sapphire layer has a thickness of less than about 500 microns. For example, the subsurface layer can be a glass having a thickness of greater than 0.2 mm, including greater than 0.3 mm or 0.4 mm, such as between about 0.3 mm to about 1.0 mm. By combining a thicker subsurface layer with a thinner sapphire layer for the cover plate formed in the method of the present invention, the composite would retain the desirable surface characteristics of the sapphire, such as hardness and scratch and smudge resistance, while also taking advantage of the desirable bulk properties of the subsurface material, such as good fracture resistance and low cost. For example, in a sapphire-glass composite structure, the sapphire would enhance the shatter and scratch resistance of the glass while, for a sapphire-polymeric material composite, the combination would be much more resistant to mechanical damage, such as cracking. Such composites would not compromise the transparency of the cover plate. Other advantageous combinations of these thin sapphire layers and transparent substrates are also possible and can be determined by one of ordinary skill in the art, given the benefit of this disclosure.

In another embodiment, the transparent layer affixed to the sapphire layer is an exterior surface coating layer. Thus, while preferably, the sapphire layer is the exterior layer of the cover plate and therefore also of an electronic device comprising the cover plate, an antireflective and/or oleophobic coating, or other desirable exterior transparent layer may also be applied to the sapphire layer. Typically this exterior transparent surface coating layer has a thickness of less than 2 microns, such as between about 0.001 microns to about 1.5 microns.

The cover plate formed in the method of the present invention may further comprise at least one transparent conducting oxide layer. This is particularly preferred when the cover plate is used for an electronic device including a capacitive touch screen in the display element in which the touch screen electrical components are integrated with the cover plate. Use of a cover plate comprising a sapphire layer produced by the method of the present invention and having a desired thickness could facilitate simpler integration of a capacitive touch screen into a display. For example, a capacitive touch screen structure in general consists of two layers of transparent conducting oxide (TCO), often separated by a dielectric layer. The two TCO layers are typically patterned into lines, with the lines on the first layer running perpendicular to the lines on the second layer, although other line patterns are also possible. The pitch of these patterned lines may be between 0.1 and 10 mm (such as 6 mm), and the width of these patterned lines may be between 0.2 and 6 mm (such as 5.9 mm or 1 mm). The dielectric layer can be a layer of glass, or, alternatively, may be a sputtered thin film, leading to a configuration having an overall thinner structure. The cover plate of the electronic device of the present invention may comprise any of these configurations of TCO layers.

The sapphire layer of the cover plate prepared by the method of the present invention have mechanical and physical properties that are desirable for use in an electronic device. For example, at room temperature, the ultrathin sapphire layer preferably has a flexural strength of at least about 700 MPA, including between about 800 and 1000 MPa, a fracture toughness (i.e., the ability of the material containing a crack or scratch to resist fracture) of greater than 1 MPa, including between about 2 and 5 MPa, a Knoop hardness of greater than about 15 GPa, including between about 17 and about 20 GPa, and/or a Vickers hardness of greater about 1000 kg/m, including between about 2000 and 3000 kg/m. The modulus, such as the Young's modulus, is also similar to the modulus of sapphire, which is typically between about 300-400 GPa, but can vary depending on the desired properties of the cover plate (such as touch sensitivity).

Thus, the present invention further relates to an electronic device comprising the cover plate described above. The electronic device can be any device known in the art comprising a display or display element, such as mobile or portable electronic devices including, but not limited to, electronic media players for music and/or video, such as an mp3 player, mobile telephones (cell phones), personal data assistants (PDAs), pagers, laptop computers, or electronic notebooks or tablets. The display element of the device may include multiple component layers, including, for example, a visual display layer such as an LCD and a touch sensitive layer as part of a touch screen application. The cover plate can be affixed to the display surface of the display element of the device or it can be a separate protective layer that can be placed or positioned over or on top of the display element and later removed if desired.

As described above, it has been found that the method of the present invention can be used to produce a sapphire layer having a surface that is smoother and more optically transparent than the initial sapphire layer from which it was prepared. For example, an initial sapphire layer is provided by cutting or sawing a wafer or layer from a sapphire boule or portion of a sapphire boule, such as a cored cylindrical section. The thickness of this layer can be reduced in using the method of the present invention by contacting with a reagent solution, as described above, and the resulting sapphire layer has been found to not only have a final surface that is smoother (i.e., Ra_{F} is less than Ra_{I}) but also the sapphire layer has been found to be significantly more transparent compared to the initial sapphire layer.

Therefore, the method of producing a sapphire layer of the present invention can also be used in a method for producing sapphire articles having surfaces with increased transparency, which is applicable in a wide variety of areas. As a particular example, when an article of sapphire, such as a boule, is prepared, as described above, it is advantageous and often necessary to analyze the article in order to identify any defects produced in the crystal, However, detection tools generally require a smooth, polished, and transparent surface in order to properly identify crystal types and flaws.

Thus, the present invention further relates to a method of producing a sapphire article, the method comprises the steps of providing an initial sapphire article having a thickness and at least one surface, the surface of the initial sapphire article having an average surface roughness value of Ra_{I}; and reducing the thickness of the initial sapphire article by contacting the surface with a reagent solution to produce the sapphire article, The sapphire article has a thickness that is less than the thickness of the initial sapphire article and further has a final surface having an average surface roughness value of Ra_{F}, wherein (Ra_{F}−Ra_{I})/Ra_{I} is less than or equal to 0.2. The sapphire article can have a variety of different shapes and/or sizes, with defined dimension based on its cross sectional shape, and the step of reducing the thickness of the sapphire article relates to reducing one or more of those dimensions, For example, the sapphire article can be a portion of a sapphire bottle, such as a sapphire brick or cored cylindrical section prepared, for example, by sawing or otherwise cutting the article from a larger sapphire boule. For this example, the step of reducing the thickness comprises removing sapphire material from at least one surface of the brick or cylindrical core, including, for example, the ends surfaces, The article may also be the sapphire boule. Furthermore, the sapphire article can also be a sapphire layer or lamina, and therefore the method is the same as the method described above. Any of the method steps, conditions, and components described above relating to producing a sapphire layer from an initial sapphire layer can also be used for the present method of producing the sapphire article from an initial sapphire article.

Furthermore, the present invention relates to a method of analyzing a sapphire article. The method comprises the steps of providing an initial sapphire article having a thickness and at least one surface, the surface of the initial sapphire article having an average surface roughness value of Ra_{I}; and reducing the thickness of the initial sapphire article by contacting the surface with a reagent solution to produce the sapphire article. The sapphire article has a thickness that is less than the thickness of the initial sapphire layer and further has a final surface having an average surface roughness value of Ra_{F}, wherein (Ra_{F}−Ra_{I})/Ra_{I} is less than or equal to 0.2. The method further comprises the step of analyzing the sapphire article. Thus, this method comprises producing a sapphire article from an initial sapphire article using the method described above following by analyzing the resulting. sapphire article. The article can be any described above, including a sapphire boule, brick, cylindrical core, or layer. The step of analyzing the sapphire article can comprise determining at least one property of the article, particular a property that is dependent on or is difficult to measure when a surface of the article is not transparent. Preferably the analysis step comprises determining the optical properties or crystal quality of the sapphire article.

The foregoing description of preferred embodiments of the present invention has been presented for the purposes of illustration and description. It is not intended to be exhaustive or to limit the invention to the precise form disclosed. Modifications and variations are possible in light of the above teachings, or may be acquired from practice of the invention. The embodiments were chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. A method of producing a sapphire article comprising the steps of:
i) providing an initial sapphire article having a thickness and at least one surface, the surface of the initial sapphire article having an average surface roughness value of Ra_{I}; and
ii) reducing the thickness of the initial sapphire article by contacting the surface with a reagent solution to produce the sapphire article,
wherein the sapphire article has a thickness that is less than the thickness of the initial sapphire article and further has a final surface having an average surface roughness value of Ra_{F}, wherein (Ra_{F}−Ra_{I})/Ra_{I} is less than or equal to 0.2.

2. The method of claim 1, wherein the initial sapphire article is a sapphire brick having a length and a width, and wherein the step of reducing the thickness of the initial sapphire article comprises removing sapphire from an end of the brick, thereby reducing the length.

3. The method of claim 2, wherein the step of providing the initial sapphire article comprises removing the sapphire brick from a sapphire boule.

4. The method of claim 1, wherein the thickness of the initial sapphire article is reduced at a rate of greater than or equal to about 10 microns/hour.

5. The method of claim 1, wherein the thickness of the initial sapphire article is reduced at a temperature of from about 250° C. to about 350° C.

6. The method of claim 1, wherein the reagent solution comprises sulfuric acid.

7. The method of claim 1, wherein the reagent solution comprises phosphoric acid.

8. The method of claim 6, wherein the reagent solution comprises phosphoric acid.

9. The method of claim 1, wherein (Ra_{F}−Ra_{I})/Ra_{I}, is less than or equal to 0.1.

10. The method of claim 1, wherein the initial sapphire article has c-axis orientation in a direction perpendicular to the initial surface.

11. The method of claim 1, wherein the initial sapphire article has an a-axis orientation in a direction perpendicular to the initial surface.

12. The method of claim 1, wherein the method further comprises the step of polishing the final surface.

13. The method of claim 1, wherein the initial sapphire article comprises single crystal sapphire prepared in a crystal growth furnace.

14. The method of claim 13, where the crystal growth furnace is a heat exchanger method furnace.

15. The method of claim 1, wherein the method further comprises the step of polishing the final surface.

16. A method of analyzing a sapphire article comprising the steps of:
i) providing an initial sapphire article having a thickness and at least one surface, the surface of the initial sapphire article having an average surface roughness value of Ra_{I}; and
ii) reducing the thickness of the initial sapphire article by contacting the surface with a reagent solution to produce the sapphire article,
wherein the sapphire article has a thickness that is less than the thickness of the initial sapphire layer and further has a final surface having an average surface roughness value of Ra_{F}, wherein (Ra_{F}−Ra_{I})/Ra_{I} is less than or equal to 0.2; and
iii) analyzing the sapphire article.

17. The method of claim 16, wherein the initial sapphire article is a sapphire brick having a length and a width, and wherein the step of reducing the thickness of the initial sapphire article comprises removing sapphire from an end of the brick, thereby reducing the length.

18. The method of claim 17, wherein the step of providing the initial sapphire article comprises removing the sapphire brick from a sapphire boule.

19. The method of claim 16, wherein the step of analyzing the sapphire article comprises determining the crystal quality of the sapphire article.
